(19) United States
(12) Patent Application Publication
(10) Pub. No.: US 2003/0064414 A1
(43) Pub. Date: Apr. 3, 2003

(54) Rapid assessment of coagulation activity in whole blood

(76) Inventors: Michael J. Benecky, Clarksville, MD (US); Keith A. Moskowitz, Rockville, MD (US); Diane R. Post, Clarksville, MD (US)

Correspondence Address: Finnegan, Henderson, Farabow,, Garrett & Dunner, L.L.P., 1300 I Street, N.W., Washington, DC, 20005-3315, US

(21) Appl. No.: 10/107,409

(22) Filed: Mar. 28, 2002

Related U.S. Application Data
(60) Provisional application No. 60/279,737, filed on Mar. 30, 2001.

(51) Int. Cl. G01N 33/567; C12Q 1/56

(52) U.S. Cl. 435/7.21; 435/13

(57) ABSTRACT

The present invention is directed to methods to rapidly assess the overall coagulant properties of a patient's blood sample by inhibiting the activation of the intrinsic contact activation pathway of coagulation and activating the extrinsic pathway of coagulation. When the sample is whole blood, the resulting clotting time represents the overall coagulant activity of the plasma and cellular components of the blood, which is indicative of existing or impending pathology arising from abnormal coagulability. The invention also provides a method for measuring the risk of a patient for a thrombotic event and for monitoring the effectiveness of procoagulant/anticoagulant therapy. A blood collection apparatus suitable for use in for performing the methods of the invention is also provided.

### DESCRIPTION OF THE INVENTION

This application claims benefit under 35 U.S.C. §119(e) to U.S. provisional application No. 60/279,737, filed Mar. 30, 2001, which is incorporated by reference herein.

### FIELD OF THE INVENTION

This invention relates generally to the fields of medical diagnostics and disease prevention. More specifically, the invention relates to diagnostic methods and test kits for rapidly assessing the coagulation activity of blood by measuring the rate of blood clotting in vitro using whole blood samples that is representative of the clotting activity in vivo. The coagulation activity in the samples of an individual's blood is an indicator of the existence or potential development of certain pathological conditions. The invention can also be used to monitor procoagulant or anticoagulant therapy of a patient.

### BACKGROUND OF THE INVENTION

Blood coagulation, or clotting, assists homeostasis by minimizing blood loss.

Blood clotting is a complex process involving multiple cellular and humoral factor initiators, cascades of activators, enzymes, and modulators, ultimately leading to the formation of fibrin, which polymerizes into an insoluble clot. Initiation of blood coagulation arises from two distinct pathways: the intrinsic (contact) and extrinsic blood clotting pathways, and are described in, for example, Davie et al., The Coagulation Cascade: Initiation, Maintenance, and Regulation, Biochemistry, vol. 30(43):10363-70 which is incorporated herein by reference.

Briefly, the intrinsic pathway is most often triggered in vitro, for example, during blood collection, when Factor XII is activated to XIa by contact with a negatively charged surface, such as a glass tube. Factor XIIa then activates a cascade in which Factor XI is activated to XIa, then Factor XIa activates Factor X to Xa. The intrinsic pathway converges into a common pathway with the extrinsic pathway when Factor X is activated. The extrinsic pathway can be initiated in vivo or in vitro when tissue factor (TF) from injured tissues or activated leukocytes, or added exogenously, comes into contact with blood and directly activates Factor VII to VIIa. The TF:Factor VIIa complex then activates zymogens Factor IX and Factor X to their enzymatically active forms, Factors IXa and Xa, respectively. Factor Xa combines with Factor Va to yield the prothrombinase complex (active procoagulant), which then cleaves prothrombin to thrombin. Thrombin, in turn, cleaves fibrinogen to produce fibrin, which forms an insoluble clot. The clot is then cross-linked by Factor XIIIa.

In vivo, clotting usually requires vessel damage, platelet activation, coagulation factors and inhibition of fibrinolysis and most often results from activation of the extrinsic physiologic pathway. Abnormal blood clotting can result in a pathological response. In fact, the propensity for blood to clot too rapidly is an important predictor of the development, progression, and recovery from a number of serious pathological conditions. Examples of such conditions include heart attack, stroke, coronary artery disease, deep vein thrombosis, and pulmonary embolism, among others. Of these diseases, coronary artery disease is the leading cause of mortality in the United States, accounting for approximately 2,500,000 deaths annually.

Furthermore, certain clinical conditions, such as vascular disease, surgery, trauma, malignancy, prosthetic vascular devices, general anesthesia, pregnancy, the use of oral contraceptives, systemic lupus erythematosus, and infection may predispose individuals to undergo adverse clotting events. Often, patients with acute conditions suspected of resulting from clotting abnormalities appear in the emergency room. A method for rapidly detecting, in a whole blood sample, the patient's current risk for clot formation would help rule in or rule out thrombotic events and coagulopathies. This would also improve the delivery of emergency health care to those who need it, while offering early identification of patients who may progress to potentially lethal clotting pathology.

Blood may also clot too slowly, or not at all, which can lead to bleeding or other blood coagulation disorders. As described in more detail below, the hemophilias are examples of inheritable congenital bleeding disorders. In addition, diseases affecting the liver, such as alcoholic cirrhosis and acute and chronic hepatitis, are associated with numerous clotting abnormalities, because this organ synthesizes many of the coagulation factors.

The best known of the inherited disorders of coagulation are hemophilia A and B, which are associated with a decrease in the activity of Factor VIII and IX, respectively. The severity of the disorder depends on the extent of depletion of the respective clotting factors. Severe cases are manifested early in life, and children with hemophilia usually show easy bleeding in large joints, such as the knees, and marked defects in clot formation. In milder forms, hemophilia may not be evident until later in life.

Treatment of hemophilias generally consists of transfusions of concentrates of blood products in which there is a large amount of coagulation Factors VIII or IX. While many hemophiliacs can lead a relatively normal life, extra precautions must be taken in engaging in sports and during surgery or dental care. Unfortunately, ten percent of people with hemophilia develop antibodies to Factor VIIIa and become refractive to treatment.

The condition in which blood clots too quickly (i.e., hypercoagulability) is also a pathological condition. Disseminated intravascular coagulation (DIC) is an example of an acquired coagulation disorder characterized by pathological clotting in which blood clots in the circulation rather than at the site of vascular injury.

Thus, a rapid and simple in vitro assessment of the overall coagulability of blood, which correlates with the risk of blood clotting in vivo, as well as the contributory effect of a particular procoagulant or anticoagulant on coagulation, would be highly informative for diagnosis, prevention, and prediction involving blood clotting disorders. Moreover, this diagnostic tool would guide the physician in selecting the appropriate therapy.

Classically, the propensity for blood to clot is determined, either manually or automatically, by measuring the time needed for a sample of plasma or blood to form insoluble fibrin strands or a clot. Clot formation may be detected visually by observing the formation of fibrin strands, or by automated methods, such as by detecting changes in viscosity by measuring mechanical or electrical impedance, or by photo-optical detection. Examples of such automated methods include the HEMOCHRON™ system (International Technidyne Corp.) or the ACTALYKE system, each of which uses a precision aligned magnet within a test tube and a magnetic detector located within the instrument to detect clot formation. Another example is the Sonoclot Coagulation and Platelet Function Analyzer (Sienco, Wheat Ridge, Colo.), which uses a disposable vibrating probe immersed in whole blood to measure the viscous drag of fibrin strands.

Other methods for the measurement of blood coagulation time that have been conventionally employed include those relying on the measurement of prothrombin time (PT), the measurement of activated partial thromboplastin time (APTT), the measurement of thrombin time, and the fibrinogen level test. Detection of a thrombotic event also may be performed by measuring the level of soluble fibrin or fibrinogen degradation products in the circulation.

The PT and APTT tests, however, are conceptually flawed. In the PT test, plasma is mixed with thromboplastin and excessive amounts of tissue factor to initiate clotting. The non-physiological amount of tissue factor significantly reduces the sensitivity of the test to factors such as Factors IX and VIII. In the APTT test, test plasma is incubated with partial thromboplastin and a highly charged surface activator such as celite, kaolin, ellagic acid, or dextran sulphate, to initiate the intrinsic pathway. The time required for a clot to form is recorded. This assay is flawed in that it is completely insensitive to abnormalities of the extrinsic pathway. Further, it is sensitive to impairment of thrombin (Factor IIa), but not Factor Xa.

Another drawback of these tests is that they are usually performed on plasma, which does not contain activated platelets and monocytes, both of which contribute significantly to normal and altered coagulation states. By excluding the influence of the cellular components of whole blood, such as monocytes, these popular plasma-based methods for measuring clotting time do not fully provide maximum predictive and diagnostic value for thrombotic events modulated by the cellular of blood.

The level of important initiators and modulators of the blood clotting process in whole blood may also be a diagnostically useful parameter for identifying patients at risk of undergoing thrombotic events. One such molecule is tissue factor, also known as Factor III, which is a transmembrane glycoprotein present on the surface of circulating cell known as monocytes. Tissue factor is also found in phospholipid vesicles within the blood plasma. Elevated levels of circulating tissue factor have been linked to many thrombotic disorders and pathologic states. Detection of elevated levels of tissue factor on circulating cells and vesicles in plasma may help to identify patients at risk for cancer, infections, and thrombotic disorders such as heart attack and stroke.

Methods for the direct measurement of tissue factor have been described. In addition to immunoassay procedures, which do not measure tissue factor activity, such as that described in U.S. Pat. No. 5,403,716, the exposure of whole blood to endotoxin, as described in U.S. Pat. No. 4,814,247 and by Spillert and Lazaro, 1993, J. Nat. Med. Assoc. 85:611-616, provide a qualitative assessment of TF levels within several hours. This assessment represents the tissue factor synthesized when the endotoxin or other immunomodulators such as interleukins creates a condition that simulates disease or trauma, thus measuring the patient's propensity to clot when experiencing such conditions. However, this test has not been standardized for the clinical setting, nor does it provide an assessment of the existing hemostatic condition or pathology of a patient.

Assays measuring the activities of other procoagulants or anticoagulants also exist. For example, the percentage of Factor XII activity present in plasma can be determined by the degree of correction obtained when the plasma is added to severely Factor XII deficient plasma. This assay is a modification of the APTT test and measures the ability of the patient's plasma to “correct” the APTT of plasma containing less than 1% Factor XII. The amount of correction achieved by dilution of the patient's plasma is compared to the correction obtained by known concentrations of Factor XII in normal plasma. Normal plasma is considered to give 100% correction.

Another assay determines the percentage of thrombin (Factor II) activity present in plasma by determining the degree of correction obtained when the plasma is added to severely Factor II deficient plasma. This assay is a modification of the prothrombin time test and measures the ability of the patient's plasma to “correct” the PT of plasma containing less than 1% Factor II. The amount of correction achieved by dilution of the patient's plasma is compared to the correction obtained by known concentrations of Factor II. Normal plasma is considered to give 100% correction by containing 100% normal levels of Factor II.

There are also immunoassays that detect markers associated with activation of the blood coagulation cascade and fibrinolysis, such as F1.2 prothrombin fragment, D-dimer, soluble fibrin, and the thrombin/antithrombin III complex. In general, these coagulation immunoassays have enjoyed limited acceptance outside the research setting since these kits involve slow and relatively labor intensive ELISA procedures.

Some of the assays described above have been used for monitoring and improving therapies involving anticoagulants, such as heparin and warfarin. The APTT assay is the most commonly used test to monitor patients undergoing anticoagulant therapy with unfractionated heparin because heparin acts on the thrombin added at the initiation of the assay. As described above, however, the APTT test does not assess clotting times that are reflective of the physiological condition because it fails to activate the extrinsic pathway. Thus, the level of heparin cannot be reliably predicted by the APTT in individual patients. Similarly, the PT test is the most commonly used assay for monitoring anticoagulant therapy involving warfarin because warfarin acts by inhibiting prothrombin that is added at the initiation of the assay. As in the case with APTT, however, the appropriate therapeutic range of warfarin varies substantially because the PT test is not reflective of the physiological condition due to the excess tissue factor needed to initiate the test.

In addition, none of the current assays are able to monitor some newer and safer anticoagulants, such as low molecular weight heparin (“LMWH”). LMWH is used both to treat existing venous thromboembolisms and to prevent such occurrences. The advantage of LMWH over unfractionated heparin is that LMWH does not cause thrombocytopenia, a serious blood disorder. Some physicians, however, are reluctant to prescribe LMWH because there is no clinically useful test available that can accurately monitor the safety of LMWH dosing. The APTT and PT tests do not accurately monitor LMWH because LMWH does not act upon thrombin or prothrombin, like unfractionated heparin and wafarin do. LMWH acts as an anticoagulant by binding primarily to Factor Xa. Moreover, as discussed earlier, neither of these assays are performed to reflect physiological conditions.

In addition to LMWH, the assays of the present invention can measure other inhibitors of coagulation. These anticoagulants include, but are not limited to, UFH, pentasaccharide, a direct thrombin inhibitor, a direct factor Xa inhibitor, a tissue factor pathway inhibitor, a factor IX inhibitor, activated protein C, or ATIII. In particular embodiments , the tissue factor pathway inhibitor TFPI, VIIai, rNAPc2, anti-TF monoclonal antibody, soluble AA mutated tissue factor, or coumadin. In other embodiments, the Factor IX inhibitor is an anti-Factor IX monoclonal antibody or FIXai.

Other assays that could be used to monitor anticoagulants include the activated clotting time (“ACT”) and chromogenic anti-Xa assays. Both of these assays, however, also have major drawbacks. The ACT, like the APTT, is measured via stimulation of the intrinsic contact activation pathway. The chromogenic assay, although an FDA-approved assay for determination of heparin and LMWH in plasma samples, is complex, inconvenient, and must normally be sent out to a laboratory before results can be reported. The time from sample collection until the lab reports back to the physician often exceeds 4 hours.

In any of these assays described above, it would be more advantageous to assay whole blood over plasma for the reasons discussed above. The use of whole blood as samples, however, has been hampered due to spontaneous coagulation via the intrinsic (contact) pathway when samples are collected into tubes. Thus, prior methods have required rapid analysis of whole blood within minutes of sampling.

One approach to bypass this problem has been to prepare plasma from the whole blood to facilitate analysis at a later time. However, as discussed above, assays using plasma do not produce accurate models of in vivo coagulation. Alternatively, one can use blood containing a calcium ion-binding anticoagulant such as citrate. In this case, the clotting time measurement is initiated by adding a calcium salt to reverse the effect of the anticoagulant. This latter determination is referred to as the recalcification time. Upon recalcification, however, whole blood may still clot due to activation of the intrinsic pathway, obscuring the true in vivo clotting mechanism that occurs via the extrinsic pathway.

It would be desirable to provide a rapid and simple in vitro assessment of the overall coagulability of whole blood that is more representative of the physiological coagulation cascade, correlates with the risk of blood clotting in vivo, and measures the contributory effect of a particular procoagulant or anticoagulant on coagulation. This would provide health care professionals with diagnostically and clinically useful data for:

(1) assessing the patient's condition; (2) selecting the proper course of therapy and dosage; and (3) monitoring and measuring the rate and effectiveness of surgical and non-surgical therapies. A rapid assessment method of overall blood coagulability that specifically evaluates the contributions of the extrinsic pathway and is representative of the initiation of in vivo coagulation was not previously available. The detection of elevated propensities to hypercoagulate or hypocoagulate will permit earlier therapy, thereby improving prognosis. The instant method can rapidly measure the hypercoaguable or hypocoaguable state by first inhibiting activation of the intrinsic coagulation pathway followed by activation of the extrinsic coagulation pathway with at least one procoagulant and measuring the coagulation time of the blood sample. This method can also be used to determine the effective dose of a particular procoagulant or anticoagulant medication, and for measuring coagulation of a patient's blood already treated with a procoagulant or anticoagulant medication.

### SUMMARY OF THE INVENTION

The present invention provides a method to rapidly assess the overall coagulant properties of a patient's blood by first inhibiting activation of the intrinsic contact activation pathway of blood coagulation followed by stimulation of the extrinsic coagulation pathway with at least one procoagulant added at a physiological concentration, and measuring the coagulation time of the blood sample, reflecting physiologic coagulation. Measurement of the coagulation time of the extrinsic pathway provides a more accurate reflection of the in vivo clotting process. Furthermore, when the sample is whole blood, the resulting clotting time represents the overall coagulant activity of the plasma and cellular components of the blood, which is indicative of existing or impending pathology arising from abnormal coagulability.

It is an object of the invention to provide a method for measuring the risk of a patient for a thrombotic event by determining the tendency of a patient's whole blood to coagulate.

It is another object of the invention to provide a method for measuring the effectiveness and safety of anticoagulant therapy, such as, for example, low molecular weight heparin (LMWH), by first inhibiting activation of the intrinsic pathway of blood coagulation followed by stimulation of the extrinsic coagulation pathway with at least one procoagulant added at a physiological concentration and comparing the coagulation time of that blood sample with that of a blood sample treated with less or no anticoagulant.

It is a further object of the invention to provide a method for determining the effective dosage of a coagulant factor (such as a procoagulant or anticoagulant) to treat a patient in need of coagulant therapy, by first inhibiting activation of the intrinsic blood coagulation pathway followed by stimulation of the extrinsic coagulation pathway with at least one procoagulant and comparing the coagulation time of that blood sample with that of a blood sample additionally treated with the coagulant factor.

Another object of the invention is to provide a method to monitor the recovery of a patient from a condition related to adverse blood coagulation by measuring the coagulation time in accordance with the methods described herein before, during, or after treatment or a surgical procedure.

It is yet another object of the invention to provide a blood collection apparatus comprising a vessel, wherein the vessel contains a contact activation pathway inhibitor. In certain embodiments, the vessel further comprises a Ca²⁺ chelator. In particular embodiments, the vessel is an evacuated tube.

It is yet another object of the invention to provide an assay apparatus comprising a vessel, wherein the vessel contains a procoagulant. In certain embodiments, the vessel further comprises a contact activation pathway inhibitor.

Additional objects and advantages of the invention will be set forth in part in the description that follows or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIGS. 1A-C illustrate the effects of inhibitors of the intrinsic contact activation pathway of blood coagulation on low molecular weight heparin (LMWH) dose response curves obtained by the present assay method. FIG. 1A illustrates that blood collection in the presence of a specific Factor XIIa inhibitor, corn trypsin inhibitor, significantly increases the slope of the LMWH dose response curve. FIG. 1B shows that blood collection in the presence of aprotinin, a kallikrein inhibitor, also increases the slope of the LMWH dose response curve. FIG. 1C shows that the effects of corn trypsin inhibitor and aprotinin are comparable to each other.

FIGS. 2A and B show dose response curves of the whole blood clotting time as assayed using the method of the present invention in response to varying doses of commercially available LMWH. FIG. 2A shows the dose response curve when Pharmacia Fragmin® was used and FIG. 2B shows the dose response curve when Aventis Lovenox® was used.

FIGS. 3A and B illustrate the correlation between whole blood clotting time as assayed using the method of the present invention and the LMWH anti-Xa activity in plasma measured by the chromogenic substrate-based ACTICHROME® Heparin anti-Xa Activity Assay. FIGS. 3A and 3B show the correlation between the two assay methods when the source of LMWH was Pharmacia Fragmin® and Aventis Lovenox®, respectively.

FIG. 4 is a diagram showing the correspondence between the plasma clotting time as assayed according to the APTT assay and the anti-Xa activity of LMWH in plasma measured by the ACTICHROME® chromogenic substrate-based assay.

FIGS. 5A and B show the correlation between the whole blood clotting times as assayed using the method of the present invention and using the activated clotting time (ACT) assay. In FIG. 5A, the ACT assay used was the Hemochron ACT reagent that stimulates the intrinsic pathway of blood coagulation with glass beads. In FIG. 5B, the ACT assay used was the Helena ACT reagent that uses celite to stimulate the intrinsic contact activation pathway of blood coagulation.

FIG. 6 is a dose response curve of the whole blood clotting time as assayed using the method of the present invention in response to varying doses of commercially available LMWH Lovenox® when blood is collected into citrate and Aprotinin and clotting is initiated with lipidated tissue factor as opposed to Factor VIIa.

FIG. 7A is a diagram showing that clotting is abolished when citrated whole blood is recalcified in the presence of high-dose (15,000 U/ml) aprotinin, demonstrating that complete contact pathway inhibition has been achieved.

FIG. 7B is a dose response curve of the whole blood clotting time as assayed using the method of the present invention in response to varying doses of commercially available LMWH Lovenox® when blood is collected into citrate alone, and contact pathway inhibition and clotting are initiated simultaneously with aprotinin and lipidated tissue factor, respectively.

FIG. 8 is a dose response curve of the whole blood clotting time as assayed using the method of the present invention in response to varying doses of commercially available LMWH Lovenox® when blood is collected into citrate alone, and contact pathway inhibition and clotting are initiated simultaneously with corn trypsin inhibitor and lipidated tissue factor, respectively.

### DESCRIPTION OF THE PREFERRED EMBODIMENTS

The overall coagulability of blood is governed by factors contributed by both the soluble (plasma) portion of blood as well as that provided by the cellular portion. Traditional measures of clotting or blood coagulability, for example, prothrombin time (PT) and activated partial thromboplastin time (APTT), among others, generally use plasma to measure blood coagulability. These plasma-based methods, however, omit contributions to blood coagulability provided by the cellular components. One example is the contribution of tissue factor to blood coagulability. As described above, tissue factor is an initiator and modulator of blood coagulation, and may be present in the blood. Elevated levels are associated with pathologic states. In addition to tissue factor, other components present in or on the cellular components of blood may also modulate blood coagulability and also contribute to the propensity for blood to clot in vivo. Thus, the practice of the present invention generally involves measuring clotting of whole blood, although blood products prepared by methods known in the art, such as plasma, platelet-deficient plasma, and reconstituted plasma can also be used.

In contrast to the above-mentioned coagulation test described by Spillert and Lazaro wherein endotoxin incubated with the whole blood sample induces the synthesis of monocyte tissue factor, which in turn influences the coagulant properties of the blood sample, the method of the present invention does not measure the effect of tissue factor synthesis on blood coagulability. Instead, it measures the influence of existing tissue factor present in the whole blood sample on blood coagulability. See, for example, Santucci et al., Measurement of Tissue Factor Activity in Whole Blood, Thromb. Haemost., vol. 83(3):445-54 (2000), which is incorporated by reference herein. Furthermore, in contrast to the PT test described above, the method of the present invention does not add a large non-physiological amount of tissue factor to initiate coagulation, but rather assesses the propensity of a patient's blood to clot at the existing or near existing level of tissue factor present in the patient's blood sample.

As discussed, the present invention provides methods for measuring coagulation of blood and blood products, and more particularly, for inhibiting the intrinsic contact pathway in vitro and stimulating activation of the extrinsic activation pathway with at least one procoagulant agent. The methods described herein allow for a significant reduction or elimination of the intrinsic pathway of coagulation and enables the measurement of blood coagulation via the extrinsic pathway, which is more representative of the in vivo state of coagulation.

In one embodiment of the present invention, an anticoagulant is added to a whole blood or blood product sample to inhibit activation of the intrinsic pathway. In a certain embodiments of the present invention, corn trypsin inhibitor or aprotinin are used to inhibit activation of the intrinsic pathway.

In another embodiment of the present invention, activation of the intrinsic pathway can be inhibited by contacting the whole blood or blood product sample with a surface having low thrombogenic activity. Examples of surfaces having low thrombogenic activity include plastic, glass, and siliconized glass. The contact can be performed during collection, storage, or handling of the whole blood or blood product sample. By the term “low thrombogenic activity” as used herein is meant having little or no blood clotting activity. Preferably, the surface having low thrombogenic activity will inhibit the whole blood or blood product sample clotting time from between about 10 to about 3600 seconds longer than a suitable control, more preferably from about 50 to about 1500 seconds, and even more preferably, from about 150 to about 1050 seconds longer than the control.

In yet another embodiment of the present invention, measurement of blood coagulation according to the methods of the invention may be performed on fresh whole blood. Alternatively, a whole blood sample may be collected in the presence of an anticoagulant that binds calcium ions, such as citrate, oxalate, etc. This does not include an anticoagulant that blocks the intrinsic pathway of clot formation, that is, the anticoagulant will block the extrinsic or common pathways. In the instance where the blood is collected with a calcium-binding anticoagulant, the effect of the anticoagulant in the blood sample must be reversed at the time blood coagulability or clotting time is measured. This is accomplished by the addition of a calcium salt, such as, for example, calcium chloride. The clotting time of a sample treated with calcium salt to reactivate the clotting process is referred to as the recalcification time. The calcium-binding anticoagulant can be added prior to or simultaneously with an anticoagulant of the intrinsic pathway.

A procoagulant of the extrinsic pathway includes recombinant tissue factor, Factor VIIa, or Factor Xa. In a preferred embodiment of the present invention, Factor VIIa is used as a procoagulant of the extrinsic pathway. Factor VIIa can be recombinant Factor VIIa or natural Factor VIIa isolated from blood. The amount of Factor VIIa added in the method of the invention is preferably in line with physiological amounts of Factor VIIa present during the in vivo initiation of the extrinsic pathway. Usually, the physiological amount of Factor VIIa is ranges from about 1 nanomole/liter to about 100 nanomoles/liter in blood, more specifically about 5 to about 100 nanomoles/liter. Thus, the in vitro measurement of clotting time according to the methods of the present invention is more representative of the in vivo clotting process.

The invention is not limited to any particular method of measuring clotting. Any number of available procedures for measuring blood clotting may be used in the present invention, including manual, semi-automated, and automated procedures, and their corresponding equipment or instruments. Instruments suitable for this purpose include, for example, all instruments that measure mechanical impedance caused by initiation of a clot. The reagents that initiate clotting or affect clotting times may be presented in various forms, including but not limited to solutions, lyophilized or air-dried forms, or dry card formats.

For example, instruments such as the HEMOCHRON™ (International Technidyne Corp.) and ACTALYKE™ (Helena Laboratories) measure clotting time using a precision aligned magnet within a test tube and a magnetic detector located within the instrument to detect clot formation. Another device, the SONOCLOT™ Coagulation Analyzer, available from Sienco, Inc., measures viscoelastic properties as a function of mechanical impedance of the sample being tested. Another device, the thrombelastograph (TEG), can also be used for measuring viscoelastic properties. An example of this type of instrumentation is the computerized thrombelastograph (CTEG), from Haemoscope Corp. The SONOCLOT™ and CTEG are capable of recording changes in the coagulation process by measuring changes in blood viscosity or elasticity, respectively. A complete graph of the entire process is obtained.

The method of the present invention for measuring the clotting time of a whole blood or blood product sample from a patient comprises the steps of inhibiting activation of the intrinsic contact activation pathway of coagulation in vitro, initiating activation of the extrinsic activation pathway of coagulation by contacting the whole blood sample with at least one procoagulant agent, and measuring the coagulation time. The methods of the invention are not adversely effected by the activity of agents, such as plasmin or tPA, or fibrinolysis inhibitors, such as, EACA or AMICAR.

In an embodiment of the invention, where the assays are performed on an emergent basis, for example, in the emergency room on a patient suspected of having an acute thrombotic event such as a heart attack or stroke, the assays may be performed directly with a fresh blood sample. The necessary reagents, including an anticoagulant of the intrinsic pathway such as corn trypsin inhibitor or aprotinin, may be preloaded into the coagulation analyzer, and at least one procoagulant of the extrinsic pathway such as Factor VIIa may also be preloaded or added subsequent to the collection of the blood sample, and the clotting times determined. Alternatively, the blood can first be collected with an anticoagulant that binds calcium ions, such as citrate, oxalate, etc. In order to reactivate clotting in a sample containing one or more of these anticoagulants, calcium salt must be added. In one embodiment of the present invention, the sample collection tube may contain an anticoagulant that binds calcium ions and an anticoagulant of the intrinsic pathway. In a separate container, calcium salt and at least one procoagulant of the extrinsic pathway may be premixed and the mixture can then be added to the whole blood sample collected in the collection tube. The time required for the formation of fibrin polymers is referred to in this instance as the recalcification time and measurement is initiated upon addition of the calcium/procoagulant mix. The difference between the recalcification time of a control sample (a sample without added procoagulant of the extrinsic pathway) versus the sample containing a procoagulant of the extrinsic pathway can be used diagnostically to indicate whether the patient has abnormal blood coagulability due to elevated tissue factor and is in need of medical intervention. In addition to the difference in clotting times, the absolute clotting times of a sample is also important and informative because a patient may be hypercoagulable due to an abnormality other than elevated tissue factor levels.

In a further embodiment of the present invention, the method of the invention can be used for monitoring anticoagulant therapy in a patient and comprises the steps of collecting blood samples from a patient undergoing anticoagulant therapy at different time points of the therapy, inhibiting activation of the intrinsic coagulation pathway, activating the extrinsic coagulation pathway with at least one procoagulant, and comparing the coagulation time of the blood samples collected at different time points. The blood samples may be analyzed individually at the time when each sample is collected or they may be stored in an anticoagulant mixture comprising an anticoagulant such as citrate and an anticoagulant of the intrinsic pathway, such as corn trypsin inhibitor, and analyzed all at the same time.

In another embodiment, the method of the invention can be used to determine the effective dose of a particular procoagulant or anticoagulant such as low molecular weight heparin (LMWH) to treat a patient having or at risk of a thrombogenic or clotting disorder. The method for determining the effective dosage of a particular procoagulant or anticoagulant for treating a patient comprises the steps of inhibiting activation of the intrinsic coagulation pathway, activating the extrinsic coagulation pathway with at least one procoagulant, and comparing the coagulation times of a patient's blood sample treated with different amounts of the particular procoagulant or anticoagulant to be used for therapy. In another embodiment, the coagulation times are compare to known ranges of effectiveness and safety.

In yet another embodiment, the present invention provides a method for monitoring the recovery of a patient from treatment or a surgical procedure attending to a condition related to adverse blood coagulation, comprising the steps of inhibiting activation of the intrinsic coagulation pathway, activating the extrinsic coagulation pathway with at least one procoagulant, and comparing the clotting times of the blood samples from the patient before, during, or after treatment or a surgical procedure. The blood samples may be analyzed individually at the time when each sample is collected or they may be stored in an anticoagulant mixture comprising an anticoagulant such as citrate and an anticoagulant of the intrinsic pathway, such as corn trypsin inhibitor, and analyzed all at the same time.

In a further embodiment of the invention, a test kit is provided for determining coagulability, comprising an anticoagulant of the intrinsic pathway at the proper concentration or a collection tube having low thrombogenic activity, and at least one procoagulant of the extrinsic pathway. In one embodiment of the present invention, the anticoagulant of the intrinsic pathway or the surface having low thrombogenic activity is separately contained from the procoagulant of the extrinsic pathway. In another embodiment, the anticoagulant of the intrinsic pathway or the surface having low thrombogenic activity is mixed with or exposed to a calcium-binding anticoagulant such as citrate. In a further embodiment, the procoagulant of the extrinsic pathway is premixed with a calcium salt to reverse the effect of the calcium-binding anticoagulant. In yet another embodiment, the contact pathway inhibitor is contained (or premixed) in the same vessel containing the procoagulant of the extrinsic pathway and a calcium salt to reverse the effect of the calcium-binding anticoagulant.

The methods of the invention provide a simple and rapid assay for measuring coagulation time. The present invention addresses the deficiencies in prior art assays. The present invention is useful and convenient for the clinical setting. The present invention allows measurement of the clotting time in vitro that is more representative of the clotting activity in vivo because it minimizes or eliminates the effects of the intrinsic contact pathway of coagulation and allows rapid measurement of the in vivo contribution of the extrinsic pathway. The present invention furthermore allows a more accurate method of monitoring procoagulant/anticoagulant therapy.

Other embodiments of the invention will be apparent to those skilled in the art from consideration of the specification and practice of the invention disclosed herein. It is intended that the specification and examples be considered as exemplary only, with the true scope and spirit of the invention being indicated by the claims.

### EXAMPLE 1

The effect of inhibitors of the intrinsic contact activation pathway of blood coagulation was assessed in this first example. The anticoagulant first used was corn trypsin inhibitor. Corn trypsin inhibitor is a specific inhibitor of Factor XIIa that selectively blocks the intrinsic contact activation pathway of blood coagulation. In contrast, corn trypsin inhibitor does not have an effect on the extrinsic tissue factor-dependent pathway of blood coagulation. Factor VIIa was used to stimulate the extrinsic pathway and clotting time was measured on whole blood samples.

Blood from a donor was drawn into two sets of collection tubes. One set was drawn into an evacuated 5 ml blood collection tube containing 0.5 ml of an anticoagulant mixture containing 3.2% sodium citrate and 500 μg/ml corn trypsin inhibitor and the other set was drawn into an identical tube, but lacking corn trypsin inhibitor. It has been well established that the anticoagulant property of sodium citrate is based on its ability to bind calcium ions required for normal blood coagulation. Each set of tubes was treated with varying doses of LMWH. Pharmacia Fragmin® was the source of LMWH used in this example. The assay was then initiated by the addition of 400 μl of each of the citrated blood samples to Hemochron P213 sample tubes (International Technidyne Corporation) that included a magnetic stir bar, 50 μl of a pH 7.4 buffered solution of Factor VIIa (26 μg/ml), HEPES (20 mM), calcium chloride (100 mM), sodium chloride (150 mM), polyethylene glycol 8000 (1 mg/ml) and bovine serum albumin (1 mg/ml). The addition of Factor VIIa to whole blood increases the levels of the TF:VIIa procoagulant complex. This leads to a Factor VIIa-concentration-dependent shortening of the whole blood clotting time upon recalcification. The levels of Factor VIIa used in this example are comparable to physiologic Factor VIIa levels present during the in vivo initiation of the extrinsic TF-dependent pathway of blood coagulation pathway.

After mixing the assay components in the Hemochron sample tubes by a brief swirling motion, the tubes were placed in a Hemochron 8000 instrument (International Technidyne Corporation) for measuring whole blood clotting time based on mechanical clot detection.

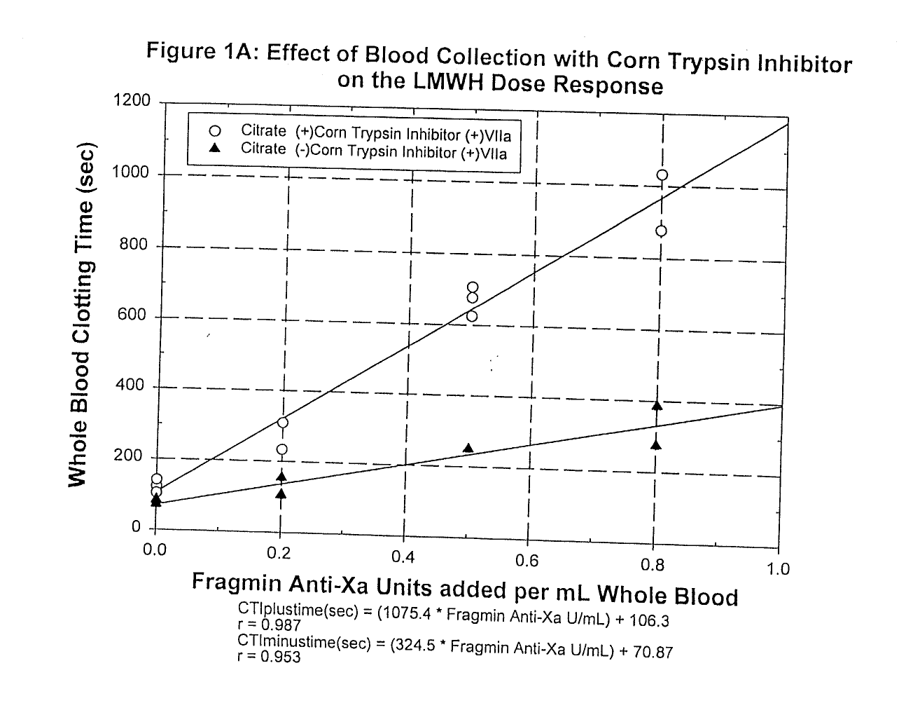

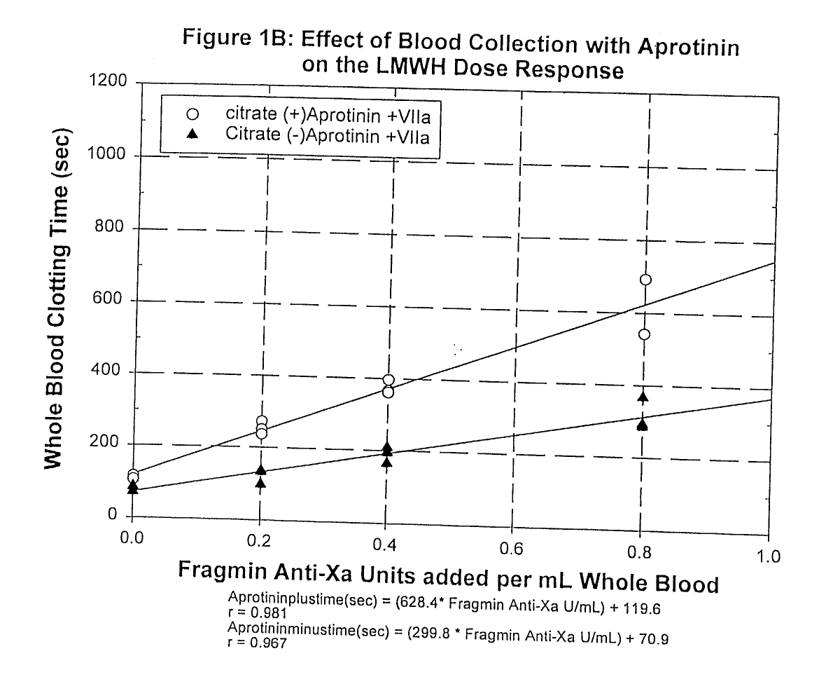

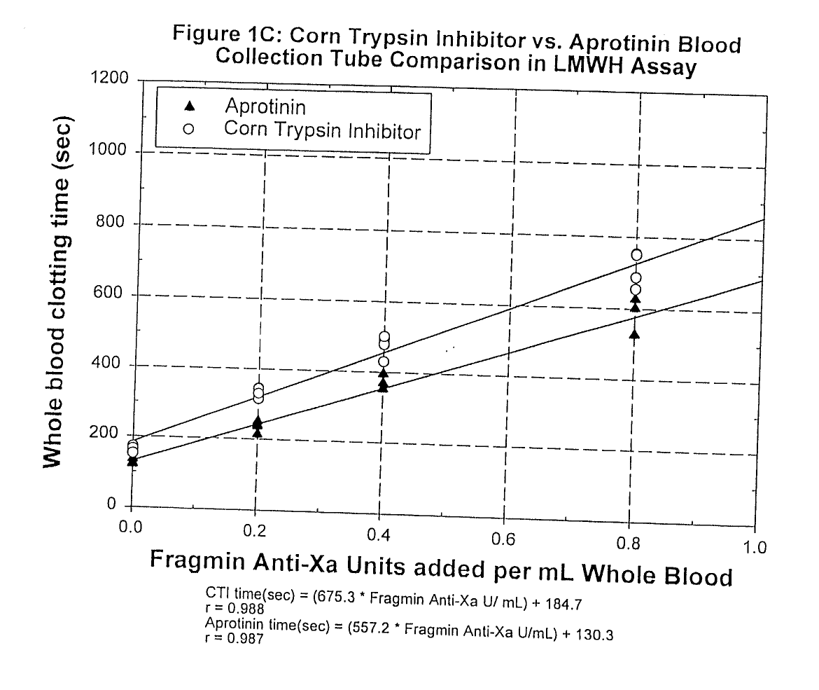

FIG. 1A shows the effect of inhibition of the intrinsic contact activation pathway of blood coagulation by corn trypsin inhibitor on blood drawn from a single individual. In the presence of corn trypsin inhibitor during blood collection and Factor VIIa to stimulate the extrinsic coagulation pathway, the slope of the Fragmin® dose response curve was approximately 1075 seconds per unit of LMWH Anti-Xa activity added. Lack of corn trypsin inhibitor during blood collection reduced the slope of the Fragmin® dose response curve approximately 3-fold to 325 seconds per unit of LMWH Anti-Xa activity added. Inhibition of the intrinsic blood coagulation pathway is reflected by the positive displacement in the corn trypsin inhibitor plus curve (open circles) relative to the corn trypsin inhibitor minus curve (solid triangles).

Another anticoagulant for inhibiting activation of the intrinsic contact pathway was compared to corn trypsin inhibitor. Aprotinin, which is also a potent plasmin inhibitor, blocks the intrinsic contact activation pathway of blood coagulation by inhibiting kallikrein.

FIG. 1B shows the effect of inhibition of the intrinsic contact activation pathway of blood coagulation by aprotinin on blood drawn from a single individual. In the presence of aprotinin during blood collection (at 100 KIU/tube) and Factor VIIa to stimulate the extrinsic tissue factor-dependent coagulation pathway, the slope of the Fragmin® dose response curve was approximately 628 seconds per unit of LMWH Anti-Xa activity added per ml of whole blood. Lack of aprotinin during blood collection reduced the slope of the Fragmin® dose response curve approximately 2-fold to 300 seconds per unit of LMWH Anti-Xa activity added per ml of whole blood. Inhibition of the intrinsic contact activation pathway is reflected by the positive displacement in the aprotinin dose response curve (open circles) relative to the citrate control curve(solid triangles).

As shown in FIG. 1C, aprotinin generated a Fragmin® LMWH dose response profile that was comparable to that obtained with corn trypsin inhibitor. Thus, aprotinin can be used as an alternative to corn trypsin inhibitor to inhibit activation of the intrinsic contact pathway.

### EXAMPLE 2

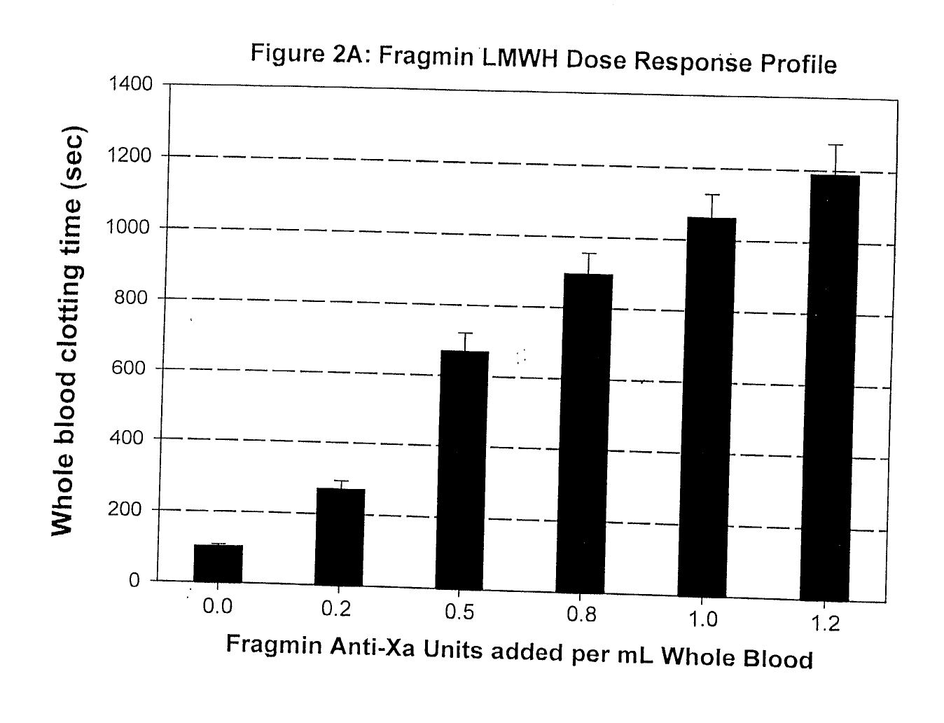

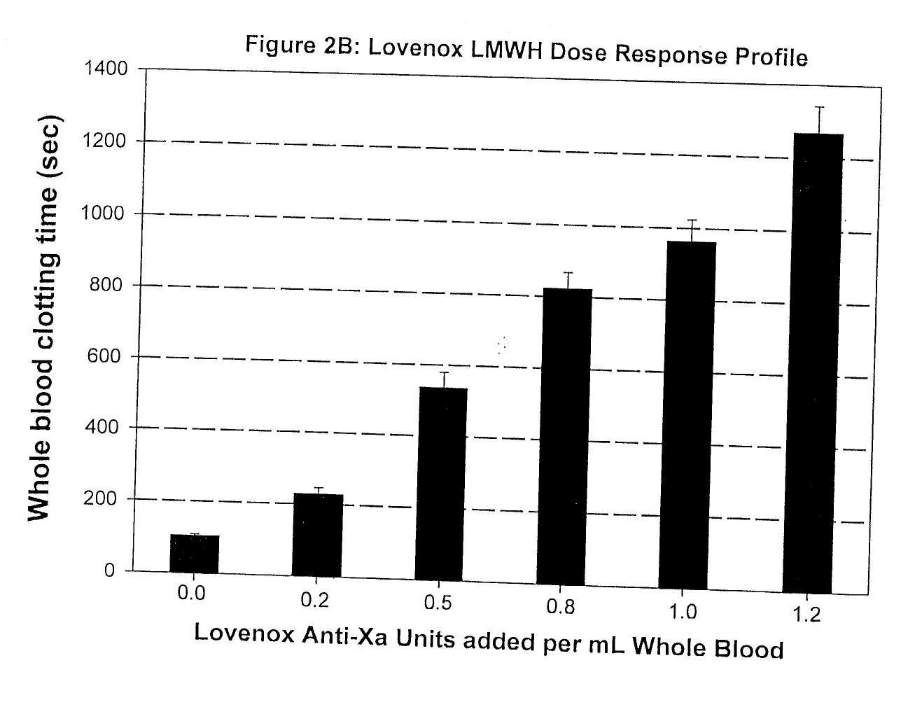

In this example, a dose response curve for LMWH was established following the method of Example 1, using two different sources of LMWH, Pharmacia Fragmin® (FIG. 2A) and Aventis Lovenox® (FIG. 2B). FIGS. 2A and 2B illustrate prolongation of the clotting time of citrate/corn trypsin inhibitor anticoagulated whole blood samples in response to progressively higher doses of LMWH. Triplicate measurements on 20 human subjects were averaged for the 0, 0.5, 0.8 and 1.0 anti-Xa U/ml LMWH levels (i.e., n=60 individual measurements). Duplicate measurements on the 20 subjects were averaged at the 0.2 and 1.2 anti-Xa U/ml LMWH levels (i.e., n=40 individual measurements). The error bars shown represent two standard errors of the mean (±2 SEM). Comparison of the effect of the two LMWHs shows that the Pharmacia Fragmin® (FIG. 2A) and Aventis Lovenox® (FIG. 2B) brands of LMWH yield similar dose response profiles.

### EXAMPLE 3

The methods for measuring coagulation exemplified in the previous examples were compared to a commercially available assay for measuring anti-Xa activity in LMWH preparations. Activity of LMWH in plasma was determined by use of the chromogenic substrate-based ACTICHROME® Heparin anti-Xa Chromogenic Activity Kit (American Diagnostica, Greenwich, Conn.). Another chromogenic assay of this type (Chromgenix, Instrumentation Laboratory) has been cleared by the FDA for the clinical determination of heparin and low molecular weight heparin in human plasma samples. However, the chromogenic assay utilizes plasma, not whole blood as in the present invention. Thus, for standardization, LMWH concentration was expressed in terms of LMWH anti-Xa units of activity measured per ml of plasma instead of LMWH anti-Xa Units of activity added per ml of whole blood. The chromogenic heparin anti-Xa assay was performed according to the manufacturer's instructions.

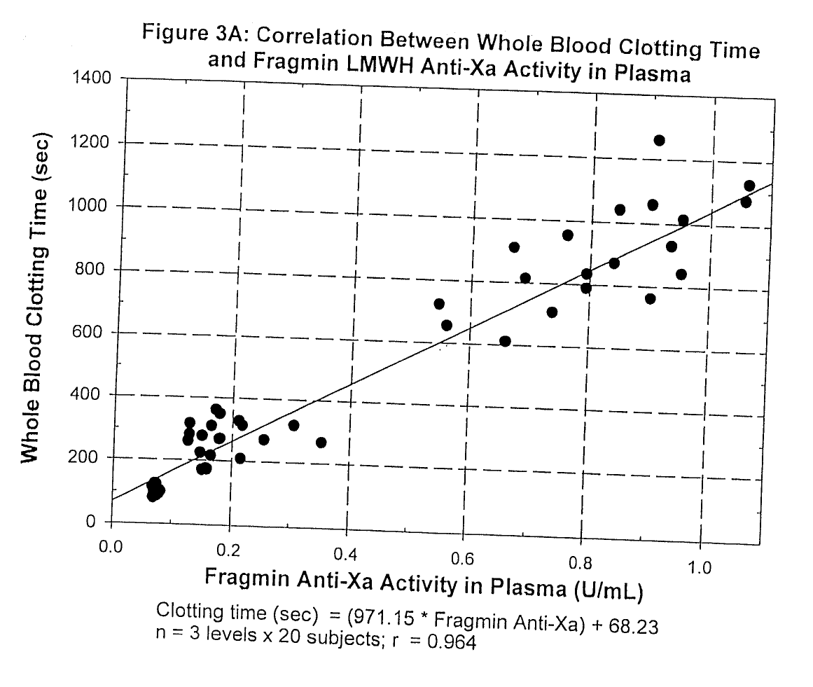

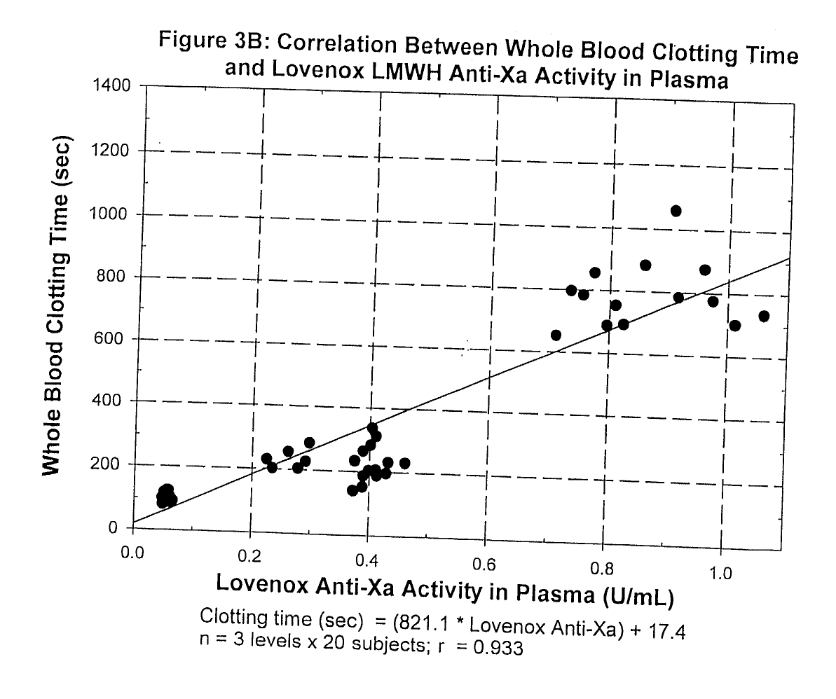

The mean whole blood clotting times for 20 healthy subjects are shown in FIGS. 3A and 3B. These plots were constructed by plotting the mean whole blood clotting times determined by the method of the invention as exemplified in Examples 1 and 2 that used corn trypsin inhibitor, and plotted against the LMWH anti-Xa activity measured in the paired plasma sample using the ACTICHROME® chromogenic substrate assay. This example shows the linear response of the assay after the in vitro addition of low (˜0.2 Anti-Xa U/ml Plasma) and high (˜0.8 Anti-Xa U/ml Plasma) levels of LMWH to whole blood.

FIG. 3A shows the excellent linear correlation (r=0.964) between the whole blood clotting time measured using the assay of the present invention and the LMWH (Pharmacia Fragmin® ) anti-Xa activity as determined using the chromogenic assay. FIG. 3B also shows a good linear correlation (r=0.9333) between the whole blood clotting time measured using the assay of the present invention and the Aventis Lovenox® LMWH anti-Xa activity as determined using the chromogenic assay.

As discussed earlier, due to its greater complexity and the requirement of plasma rather than whole blood, samples for the chromogenic assay must be normally sent out to a laboratory before results can be reported. In contrast, the present invention would be adaptable to LMWH monitoring directly in the surgical suite during cardiovascular surgical procedures on whole, unprocessed blood.

### EXAMPLE 4

In contrast to the assay of the present invention, currently popular methods of monitoring LMWH do not provide good correlation with the chromogenic assay. In this example, the activated partial thromboplastin time (APTT) method, which is currently the most commonly used method for monitoring unfractionated heparin was compared to the chromogenic assay as used in the previous example.

As in the chromogenic assay, the APTT utilizes plasma, not whole blood as in the present invention. Thus, for standardization, LMWH concentration was again expressed in terms of LMWH anti-Xa units of activity measured per ml of plasma instead of LMWH anti-Xa Units of activity added per ml of whole blood. Both assays were performed according to the manufacturer's instructions. This experiment employed the commercially available automated aPTT reagent from Organon Technika (Durham, N.C.).

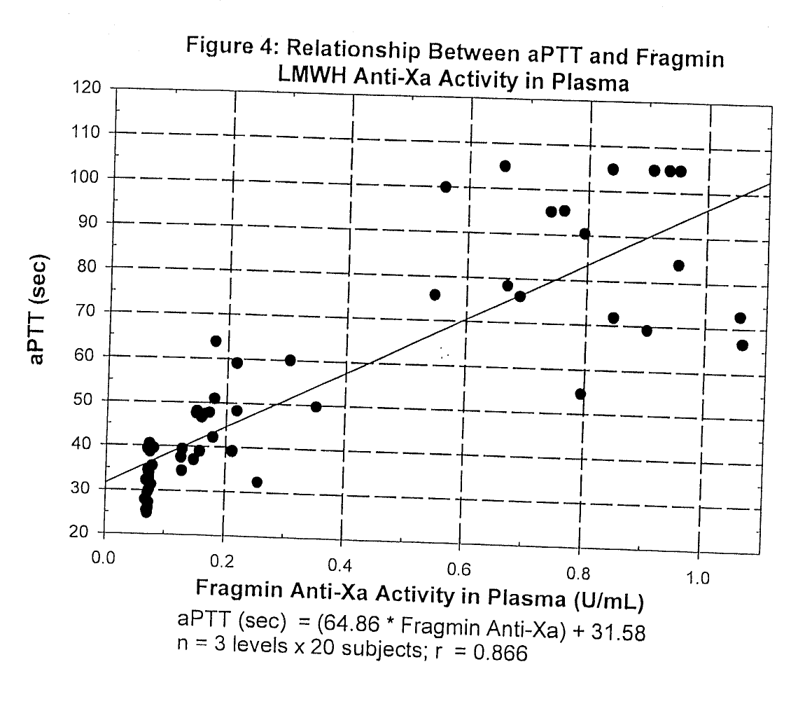

Plasma APTT values were measured on the paired plasma samples from the same group of 20 healthy subjects used to generate the LMWH anti-Xa plasma activity data shown in FIG. 3. FIG. 4 shows the correlation between APTT and LMWH anti-Xa activity in plasma for this group of 20 healthy subjects. Comparison of present whole blood assay results (FIG. 3A) with the APTT data (FIG. 4) shows that the present invention more clearly discriminates between the native unspiked (˜0.08 Anti-Xa Units/ml plasma), low level spiked LMWH (˜0.2 Anti-Xa U/ml plasma) and high level spiked (˜0.8 Anti-Xa U/ml plasma) LMWH blood samples relative to the APTT method. This result clearly illustrates the efficacy of the present invention for monitoring LMWH levels in whole blood.

### EXAMPLE 5

In the previous examples, commercially available assays that require plasma as samples were compared to the assay of the present invention. This example illustrates the superiority of the assay of the present invention for monitoring LMWH compared to a currently available assay that uses whole blood. The ACT assay is used primarily to monitor the efficacy of anticoagulant therapy in clinical procedures such as percutaneous transluminal coronary angioplasty (PTCA) or cardiopulmonary bypass surgery that involve administration of high doses of unfractionated heparin. The disadvantage of the ACT assay is the activation of the intrinsic pathway of blood coagulation by negatively charged reagents such as glass beads, celite or kaolin. Under physiological conditions, the tissue factor:Factor VIIa complex (TF:VIIa) is thought to initiate the extrinsic pathway of blood coagulation upon vascular injury.

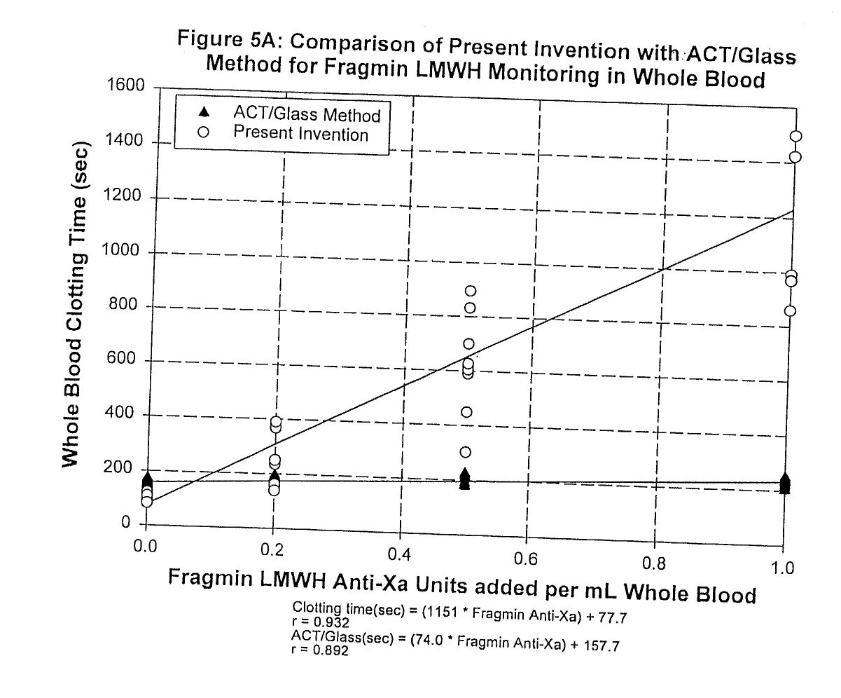

Experiments were performed using two different commercially available ACT assays and results were compared to whole blood clotting times as determined by the methods exemplified in Examples 1 and 2. In the first experiment, the Hemochron ACT reagent from International Technidyne Corporation was employed. The Hemochron ACT assay uses glass beads to stimulate the intrinsic contact activation pathway of blood coagulation. The results shown in FIG. 5A were obtained by performing assays with varying amounts of Fragmin® LMWH added to whole blood samples. Results from four individual human subjects are summarized in this graph. The data clearly shows that the present invention yields a slope for its corresponding dose response curve that is approximately 10-fold greater than the slope of the Hemochron ACT dose response profile. This observation clearly illustrates the greater sensitivity of the present invention compared to the Hemochron ACT/glass bead method for monitoring low molecular weight heparin levels in whole blood.

In the second experiment, the ACT/celite reagent from Helena Laboratories was tested and compared against the assay of the present invention. The Helena reagent uses celite to stimulate the intrinsic contact activation pathway of blood coagulation. The results shown in FIG. 5B were again obtained by performing the assays with varying amounts of Fragmin® LMWH added to the blood of an individual human subject. The data again clearly shows that the present invention yields a slope for its corresponding dose response curve that is approximately 10-fold greater than the slope of the Helena ACT dose response profile. This observation clearly illustrates the greater sensitivity of the present invention compared to the Helena ACT/celite method for monitoring low molecular weight heparin levels in whole blood.

### EXAMPLE 6

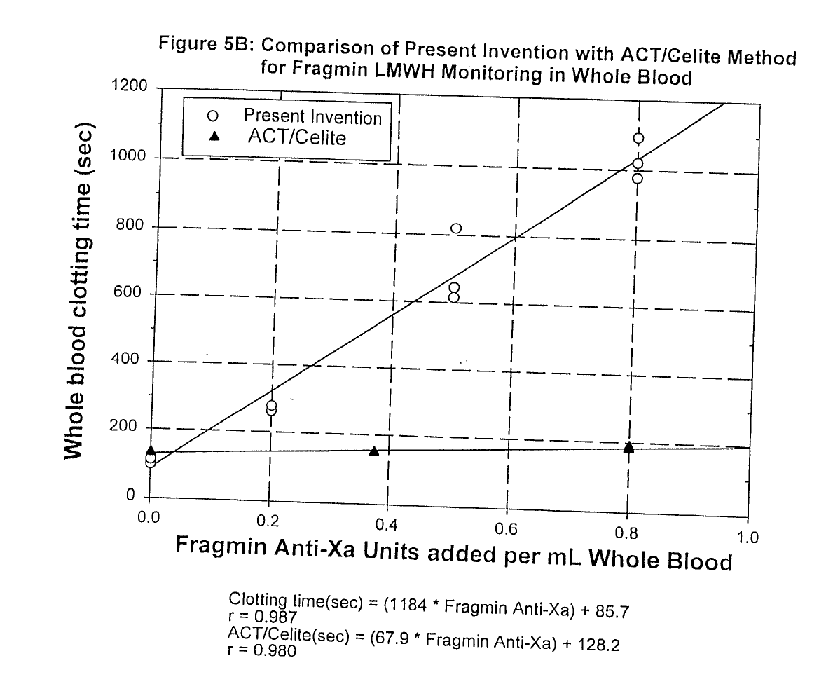

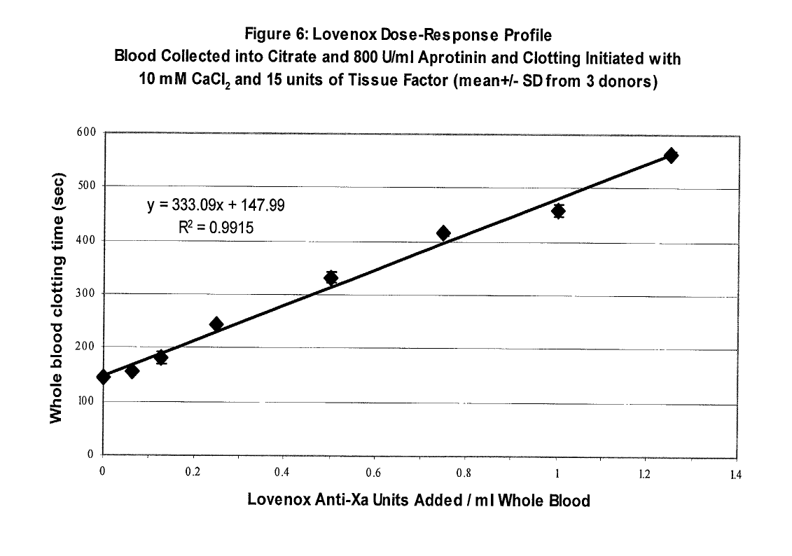

In the previous examples, clotting was initiated with plasma-derived factor VIIa. This example illustrates that coagulation may be initiated with lipidated recombinant tissue factor (Hemoliance® RecombiPlasTin, Instrumentation Laboratory Company, Lexington, Mass.). The potential advantages of lipidated tissue factor is that exposure of extravascular tissue factor initiates clotting in vivo. Thus, the system more closely resembles physiologic coagulation. Secondly, the risk of viral contamination is eliminated since the tissue factor is recombinant and the phospholipids synthetic. Lastly, Hemoliance® RecombiPlasTin is available at a fraction of the cost of plasma-derived VIIa. In this embodiment, tissue factor activity is referred to in arbitrary units based upon titration in a dilute prothrombin time (PT) assay as described by Holscermann et al., Thromb Haemost 1999; 82:1614-20, in which activity units are inversely proportional to the clotting time. For these experiments, blood was collected by syringe into 1/10 volume of 3.2% unbuffered sodium citrate containing 8000 Units/ml aprotinin (Calbiochem, LaJolla, Calif.). 50 μl of lipidated tissue factor (15 units) in the buffer described under example 1 were placed in blank MAX-ACT ACT Tubes (Helena Laboratories, Beumont, Tex.). 400 μl of blood supplemented with saline (control) or 0.06-1.2 units/ml Lovenox® were added and the clotting times recorded using an Actalyke (Helena) or Hemochron instrument. The results show that a linear relationship of clotting time in response to LMWH (R²=0.9915), a slope of 333, and intercept of 148 seconds (FIG. 6). Blood collected into citrate without aprotinin and assayed under identical conditions portrayed a slope of 140, intercept of 103 and R²=0.883 (not shown). Thus, the sensitivity and dynamic range of the present invention is improved at least two-fold relative to blood collected in the absence of aprotinin.

### EXAMPLE 7

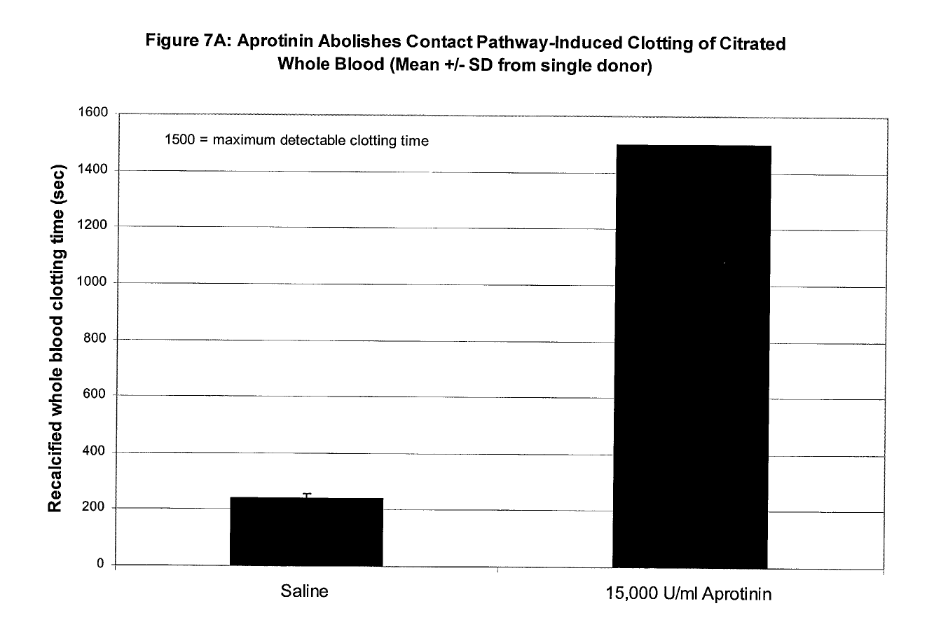

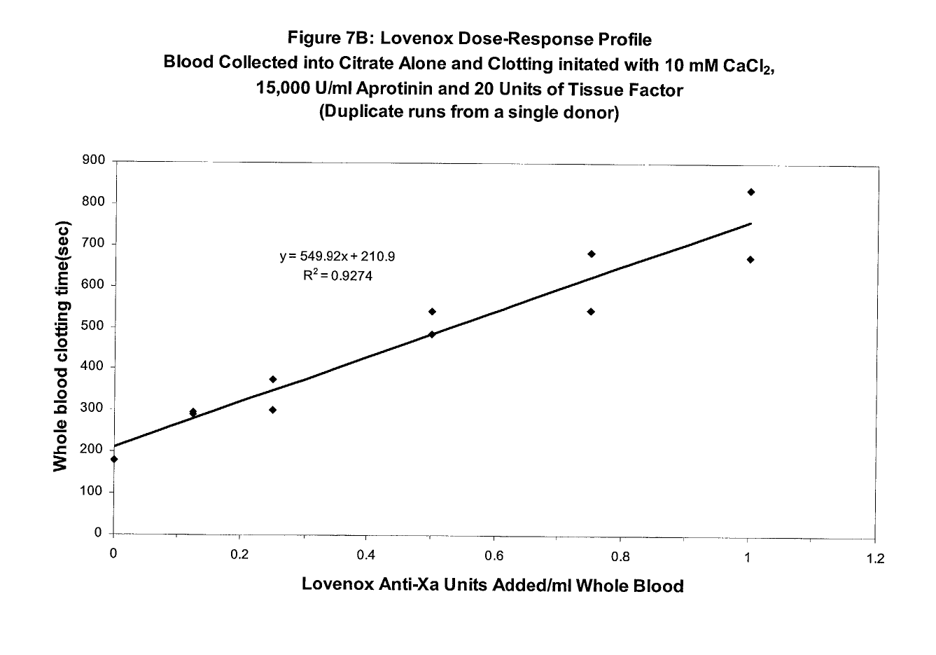

In the previous examples, blood was always collected in the presence of a calcium-chelating anticoagulant (citrate) and contact pathway inhibitor (aprotinin or corn trypsin inhibitor). It would be desirable for certain applications, for example hospital emergency department use, if blood could be clotted into a standard commercially available and FDA-approved sodium citrate tube, such as that supplied by Becton Dickinson, Franklin Lakes, N.J. and Greiner America, Monroe, N.C. and the contact pathway inhibitor added concomitantly during assay initiation with lipidated tissue factor and divalent calcium. In this example, blood from a single donor was collected into sterile evacuated-tubes containing 3.2% buffered sodium citrate (Greiner). In FIG. 7A, clotting was initiated with calcium in the presence or absence of 15,000 KIU/ml aprotinin (Centerchem Inc., Norwalk, Conn.). The results show that when blood was added to assay tubes containing calcium and aprotinin that no clotting occurred within the 1500 sec maximal clotting time of the ACT instruments. In contrast, blood recalcified in the absence of aprotinin clotted in 250 sec. Thus, the contact pathway of blood coagulation has been abolished. FIG. 7B depicts a dose-response to Lovenox® when citrated blood was recalcified in the presence of 15,000 U/ml aprotinin and 10 units of lipidated tissue factor. Notably, the addition of lipidated tissue factor reduces the recalcified clotting time from undetectable

(>1500 sec) to 180 sec. The correlation (R²) of clotting time to Enoxaparin® dose was 0.9915. The slope was 550 and intercept 210 sec. In particular, this embodiment of the described invention was sensitive to whole blood concentrations <0.1 U/ml Enoxaprin®.

### EXAMPLE 8

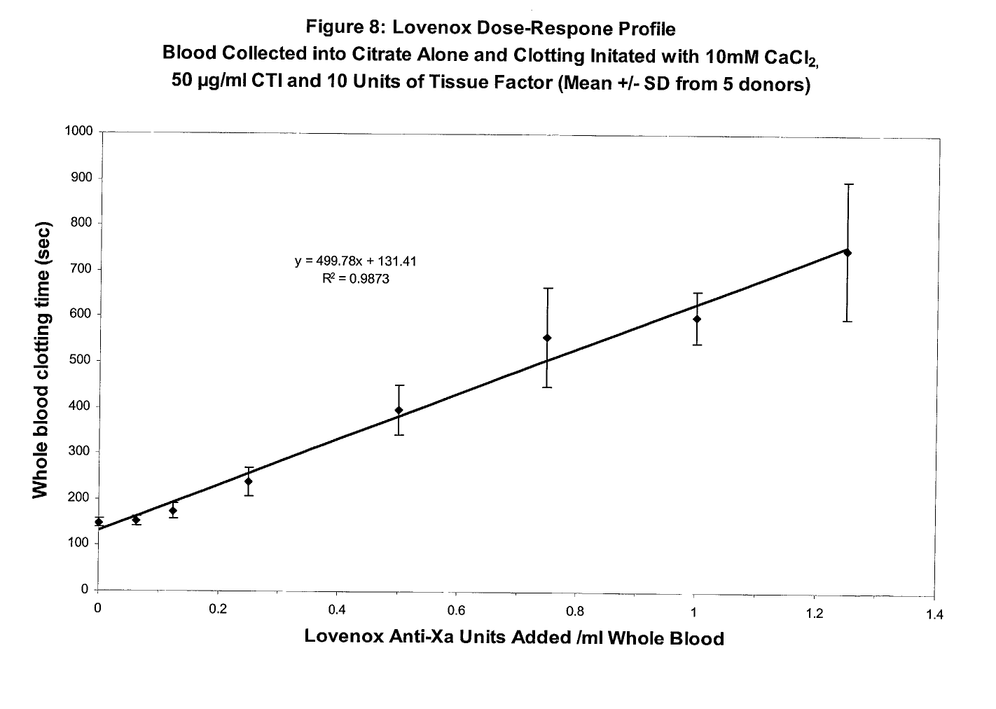

In Example 7, blood was collected into citrate alone while contact pathway inhibition and the initiation of coagulation were accomplished simultaneously upon recalcificaiton with Aprotinin and lipidated tissue factor, respectively. In this example, corn trypsin inhibitor is used to suppress contact activation under conditions where citrated blood is added to blank ACT assay tubes containing calcium, an extrinsic pathway initiating agent (lipidated tissue factor) and contact pathway inhibitor (corn trypsin inhibitor). Blood form 5 normal subjects were studied. Citrated blood (400 μl) was added to assay tubes containing 50 μl of 450 μg/ml corn trypsin inhibitor (50 μg/ml final) and 10 units of lipidated tissue factor in the buffer described under Example 1, and the clotting times recorded. The results, depicted in FIG. 8, show a linear relationship between whole blood clotting time and the Lovenox® blood concentration (R²=0.988), a slope of 500 and intercept of 130 sec. The combined results of FIGS. 7 and 8 demonstrate that in this embodiment of the described invention that adequate contact pathway inhibition can be achieved post blood draw and simultaneously upon clot initiation with calcium and lipidated tissue factor. Notably, the detection of pharmacological concentrations of the anticoagulant Enoxaparin® is not compromised and thereby allowing the use of a standard citrate evacuated blood collection tube readily available in every hospital and clinical laboratory.

While the foregoing specification teaches the principles of the present invention, with examples provided for the purpose of illustration, it will be appreciated by one skilled in the art from reading this disclosure that various changes in form and detail can be made without departing from the true scope of the invention.

We claim:

1. A method for measuring coagulation of blood, comprising
obtaining blood from a mammal;
inhibiting in vitro activation of the intrinsic contact activation pathway of coagulation in the blood;
initiating activation of the extrinsic activation pathway of coagulation by contacting the blood with at least one procoagulant; and
measuring coagulation of the blood.

2. The method according to claim 1, wherein the blood is contacted with a surface of low thrombogenic activity.

3. The method according to claim 2, wherein the low thrombogenic activity surface is plastic or siliconized glass.

4. The method according to claim 1, wherein inhibiting activation of the intrinsic contact activation pathway of coagulation comprises contacting the blood with at least one contact activation pathway inhibitor.

5. The method according to claim 4, wherein the contact activation pathway inhibitor is a Factor XIIa inhibitor, a Factor XIa inhibitor, or a kallikrein inhibitor.

6. The method according to claim 5, wherein the Factor XIIa inhibitor is corn trypsin inhibitor, an antibody to Factor XIIa, CI-esterase inhibitor, or a XIIa-binding peptide.

7. The method according to claim 5, Wherein the kallikrein inhibitor is aprotinin, an antibody to kallikrein, CI-esterase inhibitor, or a kallikrein-binding peptide.

8. The method according to claim 1, wherein the procoagulant is Factor VIIa, Factor IXa, Factor Xa, Factor XIa, viper venom, lipidated tissue factor, apo-tissue factor, or recombinant soluble tissue factor.

9. The method according to claim 8, wherein the Factor VIIa is added at a final concentration ranging from about 5 nanomoles/L to 100 nanomoles/L in the blood.

10. The method according to claim 8, wherein the Factor VIIa is recombinant Factor VIIa, natural Factor VIIa, or lipidated Factor VIIa.

11. The method according to claim 1, wherein inhibiting activation of the intrinsic contact activation pathway of coagulation comprises contacting the blood with a surface having a low thrombogenic activity, and wherein initiating activation of the extrinsic activation pathway of coagulation comprises contacting the blood with Factor VIIa, Factor IXa, Factor Xa, Factor XIa, viper venom, lipidated tissue factor, apo-tissue factor, or recombinant soluble tissue factor.

12. The method according to claim 1, wherein inhibiting activation of the intrinsic contact activation pathway of coagulation comprises contacting the blood with corn trypsin inhibitor and wherein initiating activation of the extrinsic activation pathway of coagulation comprises contacting the blood with Factor VIIa, Factor IXa, Factor Xa, Factor XIa, viper venom, lipidated tissue factor, apo-tissue factor, or recombinant soluble tissue factor.

13. The method according to claim 1, wherein inhibiting activation of the intrinsic contact activation pathway of coagulation comprises contacting the blood with aprotinin and wherein initiating activation of the extrinsic activation pathway of coagulation comprises contacting the blood with plasma or recombinant Factor VIIa, Factor IXa, Factor Xa, Factor XIa, viper venom, thrombin, lipidated tissue factor, apo-tissue factor, or soluble recombinant tissue factor.

14. The method according to claim 1, wherein inhibiting activation of the intrinsic contact activation pathway of coagulation comprises contacting the blood with CI-esterase inhibitor and wherein initiating activation of the extrinsic activation pathway of coagulation comprises contacting the blood with plasma or recombinant Factor VIIa, Factor IXa, Factor Xa, Factor XIa, viper venom, thrombin, lipidated tissue factor, apo-tissue factor, or soluble recombinant tissue factor.

15. The method according to claim 12, wherein inhibiting activation of the intrinsic contact activation pathway of coagulation comprises contacting the blood with aprotinin, and wherein initiating activation of the extrinsic activation pathway of coagulation comprises contacting the blood with Factor VIIa, wherein the Factor VIIa is natural, recombinant, or lipidated.

16. The method according to any one of claims 11 to 14, and wherein initiating activation of the extrinsic activation pathway of coagulation comprises contacting the blood with Factor Xa.

17. The method according to any of claims 11 to 14, wherein initiating activation of the extrinsic activation pathway of coagulation comprises contacting the blood with recombinant tissue factor.

18. The method according to any of claims 11 to 14, wherein initiating activation of the extrinsic activation pathway of coagulation comprises contacting the blood with lipidated tissue factor.

19. The method according to any of claims 11 to 14, wherein initiating activation of the extrinsic activation pathway of coagulation comprises contacting the blood with Factor VIIa.

20. The method according to claim 1, further comprising adding at least one anti-platelet agent to the blood.

21. The method according to claim 20, wherein the anti-platelet agent is aspirin, NSAIDs, dipyridamole, ticlopidine, clopidogrel, adenosine, theophylline, or a glycoprotein IIb/IIIa antagonist.

22. The method according to claim 1, further comprising the step of comparing coagulation of the blood to reference data defining a range of normal coagulation.

23. The method according to claim 1, further comprising the step of comparing coagulation of the blood to coagulation of a control sample, wherein the control sample has been treated with a known amount of a coagulation factor or inhibitor.

24. The method according to claim 1, wherein the blood has not been treated to prevent clotting, or has been citrated and recalcified.

25. The method according to claim 1, wherein inhibition of the intrinsic contact activation pathway occurs concurrently with activation of the extrinsic activation pathway.

26. The method according to claim 25, wherein the blood has not been treated to prevent clotting, or has been citrated and recalcified.

27. The method according to claim 5, wherein the Factor XIa inhibitor is an antibody to factor XI, CI-esterase inhibitor, or a Factor XIa-binding peptide.

28. The method according to claim 27, wherein the antibody is a monoclonal antibody.

29. The method according to claim 21, wherein the glycoprotein IIb/IIIa antagonist is abciximab, eptifibatide, or tirofiban.

30. The method according to claim 1, wherein the blood has been treated with low molecular weight heparin, UFH, pentasaccharide, a direct thrombin inhibitor, a direct factor Xa inhibitor, a tissue factor pathway inhibitor, a Factor IX inhibitor, activated protein C, or ATIII.

31. The method according to claim 21, wherein a coagulation inhibitor is administered to the mammal before the blood is obtained.

32. The method according to claim 30, wherein the coagulation inhibitor is low molecular weight heparin, UFH, pentasaccharide, a direct thrombin inhibitor, a direct factor Xa inhibitor, a tissue factor pathway inhibitor, a Factor IX inhibitor, activated protein C, or ATIII.

33. A method for measuring the effectiveness of at least one coagulation factor or coagulation inhibitor on the coagulation of blood, comprising
obtaining blood from a mammal;
dividing the blood into at least two aliquots;
treating the first aliquot to steps comprising:
(a) inhibiting in vitro activation of the intrinsic contact activation pathway of coagulation;
(b) initiating activation of the extrinsic activation pathway of coagulation by contacting the first aliquot with a procoagulant; and
(c) measuring coagulation of the first aliquot;
treating the second aliquot to steps comprising:
(d) inhibiting in vitro activation of the intrinsic contact activation pathway of coagulation in vitro;
(e) contacting the second aliquot with the at least one coagulation factor or coagulation inhibitor;
(f) initiating activation of the extrinsic activation pathway of coagulation by contacting the second aliquot with at least one procoagulant; and
(g) measuring coagulation of the second aliquot; and
(h) comparing coagulation measurements of the first and second aliquots.

34. A method for measuring the effectiveness of at least one coagulation factor or coagulation inhibitor on coagulation of a blood sample, comprising
obtaining a first blood sample from a mammal;
inhibiting activation of the intrinsic contact activation pathway of coagulation;
initiating activation of the extrinsic activation pathway of coagulation by contacting the first blood sample with a procoagulant agent;
measuring coagulation of the first blood sample;
obtaining a second blood sample from the mammal;
inhibiting activation of the intrinsic contact activation pathway of coagulation;
contacting the second blood sample with at least one coagulation factor or inhibitor;
initiating activation of the extrinsic pathway of coagulation by contacting the second blood sample with at least one procoagulant;
measuring coagulation of the second blood sample; and
comparing coagulation measurements of the first and second blood samples.

35. The method according to claim 33, wherein the coagulation factor or coagulation inhibitor is administered to the mammal before the second blood sample is obtained.

36. The method according to any one of claims 33 or 34, further comprising adjusting the concentration of the at least one coagulation factor or coagulation inhibitor in the mammal after the coagulation measurements are compared.

37. The method according to any one of claims 33 or 34, further comprising administering at least one second coagulation factor or coagulation inhibitor to the mammal after the coagulation measurements are compared.

38. The method according to claim 33, wherein the blood has not been treated to prevent clotting, or has been citrated and recalcified.

39. The method according to claim 34, wherein the blood has not been treated to prevent clotting, or has been citrated and recalcified.

40. The method according to claim 4, wherein the procoagulant is lipidated tissue factor and the contact activation pathway inhibitor is aprotinin.

41. A blood collection apparatus comprising a vessel, wherein the vessel contains a contact activation pathway inhibitor.

42. The apparatus of claim 41, wherein the vessel is an evacuated tube.

43. The apparatus of claim 41, further comprising a Ca²⁺chelator.

44. A method for monitoring recovery of a patient from a condition related to abnormal blood coagulation, comprising:
obtaining at least two blood samples from a patient;
inhibiting activation of the intrinsic contact activation pathway of coagulation in the blood samples;
initiating activation of the extrinsic activation pathway of coagulation by contacting the blood samples with at least one procoagulant; and
measuring coagulation of the blood, wherein one of the blood samples is obtained before administration of medical treatment or a surgical procedure and the other blood samples are obtained during or after administration of the medical treatment or the surgical procedure.

45. The method of claim 33, wherein the coagulation inhibitor is low molecular weight heparin, UFH, pentasaccharide, a direct thrombin inhibitor, a direct factor Xa inhibitor, a tissue factor pathway inhibitor, a Factor IX inhibitor, activated protein C, or ATIII.

46. The method of claim 45, wherein the tissue factor pathway inhibitor is TFPI, VIIai, rNAPc2, anti-tissue factor monoclonal antibody, soluble AA mutated tissue factor, or coumadin.

47. The method of claim 46, wherein the Factor IX inhibitor is an anti-Factor IX monoclonal antibody or FIXai.

48. The method of claim 34, wherein the coagulation inhibitor is low molecular weight heparin, UFH, pentasaccharide, a direct thrombin inhibitor, a direct factor Xa inhibitor, a tissue factor pathway inhibitor, a Factor IX inhibitor, activated protein C, or ATIII.

49. The method of claim 48, wherein the tissue factor pathway inhibitor is TFPI, VIIai, rNAPc2, anti-tissue factor monoclonal antibody, soluble AA mutated tissue factor, or coumadin.

50. The method of claim 49, wherein the Factor IX inhibitor is an anti-Factor IX monoclonal antibody or FIXai.
